(19) United States
(12) Patent Application Publication
(10) Pub. No.: US 2006/0252058 A1
(43) Pub. Date: Nov. 9, 2006

(54) Chip for detection of nucleic acid

(75) Inventors: Toshizo Hayashi, Tokyo (JP); Yasumasa Mitani, Hiroshima (JP)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Kabushiki Kaisha DNAFORM

(21) Appl. No.: 11/318,048

(22) Filed: Dec. 23, 2005

(30) Foreign Application Priority Data
Mar. 18, 2004 (JP) 2004-078890

(51) Int. Cl. C12Q 1/68; G06F 19/00; C12P 19/34; C12M 1/34

(52) U.S. Cl. 435/6; 435/91.2; 435/287.2; 702/20

(57) ABSTRACT

A testing chip for detecting a target nucleic acid from a liquid sample which at least comprises a support, a sample introduction part provided on the support, and a test region wherein an amplification reagent for amplifying the target nucleic acid is immobilized in the inside or on the surface of the support.

### TECHNICAL FIELD

The present invention relates to a chip for detection of a nucleic acid which can be used for detection of a target nucleic acid from a sample in a genetic testing.

### BACKGROUND ART

The genetic testing is effective as a diagnosis method of diseases or disorders, and various techniques are in practical use in clinical places. As such technique, a gene cloning method, a southern blotting method, an amplification method for a nucleic acid such as polymerase chain reaction (PCR), a hybridization method and the like are known to be used.

The methods as described above generally include a plurality of complicated processes. For example, in the southern blotting method, it is necessary to pretreat a sample and then to isolate a DNA by electrophoresis, followed by its detection. In the PCR method, it is necessary to pretreat a sample, to carry out an amplification reaction of a nucleic acid, to subject the reaction product to electrophoresis, and then to detect an amplification product. In the hybridization method, it is necessary to denature the nucleic acid contained in the sample by heat treatment or alkali treatment to give single strands, immobilize this single-stranded nucleic acid onto a solid phase carrier, and then to carry out hybridization of the immobilized nucleic acid with a labeled probe.

In addition, these steps are usually carried out using a plurality of vessels or devices, and in many cases, they are carried out in a plurality of areas in the laboratory. Accordingly, a biological sample or a reagent in a genetic testing needs to be moved to another vessel, or transported to another area, which leads to a problem of contamination of the sample by other clinical samples or amplification products, and contamination of other samples by scattering, aerosolization or the like of the sample. Furthermore, since it is unclear if any pathogen is contained in the sample, sufficient caution is needed in its handling. In addition, the genetic testing is carried out using special and expensive apparatuses and devices in many cases. Furthermore, in the case where many samples are treated at the same time, the samples are likely mixed up.

In the past, several attempts have been made to solve these problems. For example, the specification of U.S. Pat. No. 5,229,297 (Patent Document 1) describes a cuvette for amplification and detection of a gene, comprising a channel that connects a sample, a reagent for amplification and a garbage part. This is constituted in such a way that a dividing wall isolating the sample from a detection reagent is broken by crushing and/or compressing the sample in a fixed direction using a special device called a roller, and the mixture thereof is extruded into the detection part and further the garbage part through the channel. The detection of a nucleic acid using this cuvette needs to use special and complicated means and vessels.

The pamphlet of WO 95/11083 (Patent Document 2) describes a disposable reaction tube which is used for nucleic acid amplification assay. This reaction tube has a cap for sealing which can be perforated such that it is possible to perforate the pipetter into the cap after an amplification reaction, and to move the sample into the detection part without opening the cap. This reaction tube prevents contamination of other samples by scattering of the sample and generation of aerosol, and further reduces possibility of false positive, but it is not excluded from risk of infection with a pathogen contained in the sample, complexity of procedures, necessity of a special device and the like.

Recently, assay kits are marketed for detection of a target nucleic acid utilizing a method of amplifying the target nucleic acid by an isothermal reaction, for example, ICAN method (Patent Document 3: the pamphlet of WO 02/16639), LAMP method (Patent Document 4: the pamphlet of WO 00/28082) and the like. In the detection of a nucleic acid using these kits, a sample needs to be pretreated, and in addition, a reaction solution needs to be developed with a substrate for chromatography after an amplification reaction of a nucleic acid is carried out.

### DISCLOSURE OF INVENTION

The present inventors have found that use of a chip wherein an amplification reagent for amplification of a target nucleic acid is immobilized on a support allows detection of the target nucleic acid present in a liquid sample. The present invention is based on this finding.

Accordingly, an object of the present invention is to provide a testing chip which is used for detecting a target nucleic acid.

And, the testing chip according to the present invention is a testing chip for detecting a target nucleic acid from a liquid sample, which at least comprises a support, a sample introduction part provided on the support, and a test region wherein an amplification reagent for amplification of the target nucleic acid is immobilized in the inside or on the surface of the support.

According to the present invention, it is possible to carry out detection of a target nucleic acid from a sample rapidly and simply, whereby even a person who is not familiar to a biological experiment can carry out a genetic testing easily at home or a bedside. In addition, the testing chip according to the present invention can be made disposable, whereby contamination by other samples can be prevented.

### BRIEF DESCRIPTION OF THE DRAWINGS

FIG. 1 is a diagram illustrating a schematic view of the action mechanism of the amplification reaction of a nucleic acid using a first primer in the isothermal amplification primer method.

FIG. 2 is a diagram illustrating an example of the structure of a second primer which is used in the isothermal amplification primer method according to the second aspect.

FIG. 3a is a diagram illustrating a schematic view of the action mechanism of the amplification reaction of a nucleic acid in the isothermal amplification primer method according to the second aspect.

FIG. 3b is a diagram illustrating a schematic view of the action mechanism of the amplification reaction of a nucleic acid in the isothermal amplification primer method according to the second aspect.

FIG. 4 is a diagram illustrating the location of the first and the second primers which are used in amplification of human STS DYS237 gene on the gene.

FIG. 5 is a diagram illustrating the testing chip in one aspect of the present invention.

FIG. 6 is a cross-sectional view of the test region in the testing chip in one aspect of the present invention.

FIG. 7 a diagram illustrating the location of the first and the second primer which are used for detection of single nucleotide polymorphism in human acetaldehyde dehydrogenase gene on the gene.

FIG. 8 is a diagram illustrating the location of the first and the second primers which are used in amplification of the target nucleic acid specific to Type B hepatitis virus.

FIG. 9 is a diagram illustrating the testing chip in one aspect of the present invention.

FIG. 10 is a conceptual view of the gene analysis method of by a preferable embodiment of the present invention.

### REFERENCE NUMERALS

501 TESTING CHIP

502 SUPPORT WHICH COMPRISES FILTER PAPER

503 TEST REGION 1

504 TEST REGION 2

505 TEST REGION 3

506 HANDLE PART

601 SUPPORT WHICH COMPRISES FILTER PAPER

602 WATER-ABSORBING MATERIAL WHEREIN AMPLIFICATION REAGENT IS IMMOBILIZED

603 MESH

901 TESTING CHIP

902 SUPPORT WHICH COMPRISES FILTER PAPER

903 PART FOR IMMOBILIZING NUCLEIC ACID EXTRACTION REAGENT

904 TEST REGION 1

905 TEST REGION 2

906 HANDLE PART

1001 TESTING CHIP ACCORDING TO THE PRESENT INVENTION

1002 SIGNAL DETECTION DEVICE

1003 PORTABLE TERMINAL

1004 INTERNET

1005 COMPUTER FOR GENE ANALYSIS

1006 INFORMATION STORAGE DEVICE

1007 INFORMATION STORAGE DEVICE

### BEST MODE FOR CARRYING OUT THE INVENTION

The testing chip according to the present invention, first, comprises a test region wherein an amplification reagent for amplification of a target nucleic acid is immobilized, wherein the test region is placed in the inside or on the surface of a support. The reagent contained in this test region is not particularly limited, but may be those which make various amplification methods for a nucleic acid known to those skilled in the art performable. The composition of the amplification reagent can be suitably determined by those skilled in the art depending on the amplification method for a nucleic acid to be used.

The “target nucleic acid” or the “sequence of the target nucleic acid” in the present invention means not only a nucleic acid for amplification or its sequence, but also a nucleic acid which has a complementary sequence to this or the mentioned sequence.

The amplification method for a nucleic acid may be a method which allows amplification of a target nucleic acid of interest from a solution containing the nucleic acid (specifically, RNA or DNA) extracted from a sample, and various methods are known as such method (generally, see D. Kwoh and T. Kwoh, Am. Biotechnol. Lab. 8, 14-25, (1990)). Suitable amplification method for a nucleic acid includes, for example, the polymerase chain reaction method (the PCR method; the specification of U.S. Pat. No. 4,683,195, the specification of U.S. Pat. No. 4,683,202, the specification of U.S. Pat. No. 4,800,159 and the specification of U.S. Pat. No. 4,965,188), the reverse transcription PCR method (the RT-PCR method; Trends in Biotechnology 10, pp. 146-152, (1992)), the ligase chain reaction method (the LCR method; the specification of EP-A No. 0320308, R. Weiss, Science 254, 1292, (1991)), the strand displacement amplification method (the SDA method; G Walker, et al., Proc. Natl. Acad. Sci. USA 89, 392-396, (1992); G Walker, et al., Nucleic Acids Res. 20, 1691-1696, (1992)), the transcription-based amplification method (D. Kwoh, et al., Proc. Natl. Acad. Sci. USA 86, 1173-1177, (1989)), the self-sustained sequence replication method (the 3SR method; J. Guatelli, et al., Proc. Natl. Acad. Sci. USA 87, 1874-1878, (1990)), the Qβ replicase method (P. Lizardi, et al., BioTechnology 6, 1197-1202, (1988)), the nucleic acid sequence-based amplification method (the NASBA method; R. Lewis, Genetic Engineering News 12(9), 1, (1992)), the repair chain reaction method (the RCR method; R. Lewis, Genetic Engineering News 12(9), 1, (1992)), the boomerang DNA amplification method (the BDA method; R. Lewis, Genetic Engineering News 12(9), 1, (1992)), the LAMP method (the pamphlet of WO 00/28082), the ICAN method (the pamphlet of WO 02/16639) and the like.

For example, in the PCR method, a thermostable DNA polymerase, one pair of oligonucleotide primers designed on the basis of the nucleotide sequences at both terminals of a target nucleic acid and a buffer solution containing dNTP and the like are usually used. Accordingly, in the case where the PCR method is used, the test region contains these reagents. In the PCR method, by repeating a reaction which comprises three steps, i.e., dissociation into single-stranded nucleic acids (denaturation) of a double-stranded nucleic acid which serves as a template, annealing of primers to the single-stranded nucleic acids, and synthesis of a complementary strand from the primers (extension), amplification of the target nucleic acid from a DNA is made possible. In this method, a total of three steps are repeated to regulate the reaction solution to a temperature suitable in each of the three steps.

Further, in the LCR method, two pairs of oligonucleotide probes are usually used, and one pair of them is bound to one strand of a target nucleic acid, and the other pair is bound to the other strand of the target nucleic acid. Each pair is completely overlapped with the corresponding strand together. For the reaction, first, a double-stranded nucleic acid is denatured (i.e., isolation) in a nucleic acid sample, and then, two pairs of oligonucleotide probes are reacted with the strand in the presence of a thermostable ligase, whereby each pair of the oligonucleotide probes is ligated, and then the reaction product is isolated. This is repeated in circulation until the sequence is amplified to a desired extent. Accordingly, in the case where the LCR method is used, the above-mentioned test region contains the two pairs of oligonucleotide probes described above, the thermostable ligase, a buffer solution and the like.

According to a preferable embodiment of the present invention, the amplification reagent composition allows amplification of a target nucleic acid under a constant temperature. Accordingly, the amplification reagent contained in the test region allows an isothermal amplification method, and such isothermal amplification method includes, for example, the 3SR method, the Qβ replicase method, the NASBA method, the SDA method, the LAMP method, the ICAN method and the like described above. Preferable isothermal amplification method includes the SDA method, the LAMP method and the ICAN method.

For example, in the SDA method, a target nucleic acid can be amplified under isothermal conditions by using one pair of amplification primers which have a recognition site of a restriction enzyme, and further another pair of bumper primers to sandwich the amplification region, i.e., a total of four primers. A nick is generated at a restriction site on the amplification primer by the restriction enzyme, and then extension synthesis is carried out from the nick by a DNA polymerase toward the 3′ side of the amplification primer, whereby the downstream complementary strand of the target strand, which was previously formed, is substituted. This step is repeated infinitely, because the restriction enzyme continuously generates nicks to a new complementary strand formed from the restriction site, and the DNA polymerase forms a new complementary strand continuously from the restriction site at which the nick is generated. Accordingly, in the case where the SDA method is used, the test region contains the four primers, the restriction enzyme, the DNA polymerase, a buffer solution and the like.

As the isothermal amplification method, the amplification method using the isothermal amplification primers developed by the present inventors (hereinafter, called the “isothermal amplification primer method”) can also be used suitably. In this method, special primers (isothermal amplification primers) are used in the amplification method for a nucleic acid using the strand displacement reaction. The first primer used in the isothermal amplification primer method comprises Sequence (Ac′) at the 3′ end portion, which hybridizes to Sequence (A) at the 3′ end portion of a target nucleic acid sequence, and also comprises Sequence (B′) at 5′ side of the Sequence (Ac′), which hybridizes to Sequence (Bc) complementary to Sequence (B) which is present closer to 5′ side than the Sequence (A) in the target nucleic acid sequence.

The “hybridization” in the present invention means that a part of the primers according to the present invention hybridizes to a target nucleic acid under stringent conditions, and does not hybridize to nucleic acid molecules other than the target nucleic acid. The stringent conditions can be decided depending on the melting temperature Tm (° C.) of the double strand of the primers according to the present invention and its complementary strands, salt concentration of the hybridization solution and the like, which can be referred to, for example, J. Sambrook, E. F. Frisch, T. Maniatis; Molecular Cloning 2nd edition, Cold Spring Harbor Laboratory (1989) and the like. For example, if hybridization is carried out at a temperature slightly lower than the melting temperature of the primers to be used, the primers can be specifically hybridized to the target nucleic acid. Such primer can be designed by using a primer construction software on the market, for example, Primer 3 (manufactured by Whitehead Institute for Biomedical Research) and the like. According to a preferable embodiment of the present invention, the primer which hybridizes to a certain target nucleic acid comprises the whole or a part of the sequence of the nucleic acid molecule which is complementary to the target nucleic acid.

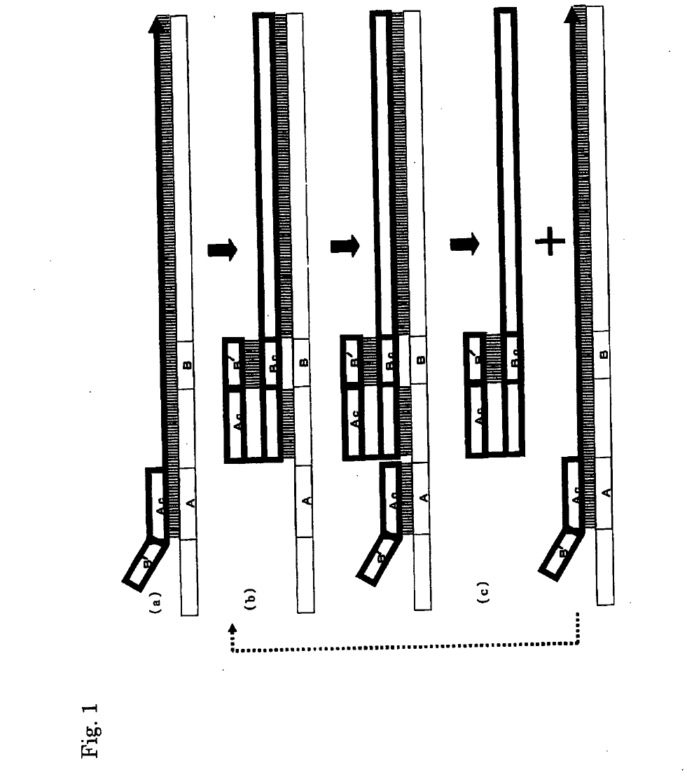

The action mechanism of nucleic acid synthesis by the first primer used in the isothermal amplification primer method is shown schematically in FIG. 1. First, a target nucleic acid sequence which serves as a template in the nucleic acid is determined, and Sequence (A) at the 3′ end portion of the target nucleic acid sequence and Sequence (B) which is present closer to 5′ side than the Sequence (A) are determined. The first primer comprises Sequence (Ac′), and further comprises Sequence (B′) on the 5′ side. The sequence (Ac′) hybridizes to the Sequence (A), and the Sequence (B′) hybridizes to the Sequence (Bc) which is complementary to Sequence (B). Here, the first primer may comprise an intervening sequence having no influence on the reaction between the Sequence (Ac′) and the Sequence (B′). If such primer is annealed to the template nucleic acid, the Sequence (Ac′) in the primer becomes hybridized to the Sequence (A) of the target nucleic acid sequence (FIG. 1(a)). If primer extension reaction takes place in this state, a nucleic acid containing a complementary sequence of the target nucleic acid sequence is synthesized. Then, the Sequence (B′) present at the 5′ terminal side of the synthesized nucleic acid hybridizes to the Sequence (Bc) present in the same nucleic acid, thereby a stem-loop structure is formed at the 5′ end portion of the synthesized nucleic acid. As a result, the Sequence (A) on the template nucleic acid becomes single-stranded, to which another primer having the same sequence with the above first primer hybridizes (FIG. 1(b)). Subsequently, extension reaction takes place from the first primer which has been newly hybridized by the strand displacement reaction, and at the same time the previously synthesized nucleic acid separates from the template nucleic acid (FIG. 1(c)).

In the action mechanism described above, the phenomenon that the Sequence (B′) hybridizes to the Sequence (Bc) typically takes place due to the presence of a complementary region on the same strand. In general, when a double-stranded nucleic acid is dissociated into single strands, partial dissociation starts from the terminal or other relatively unstable parts. In the double-stranded nucleic acid produced in the extension reaction by the first primer, the base pairs at the end portions are in an equilibrium state between dissociation and binding at relatively high temperature, keeping double-strandedness as a whole. If a sequence complementary to the dissociated part at the terminal is present on the same strand in such a state, a stem-loop structure can be formed as a metastable state. Although this stem-loop structure is not present stably, the same other primer binds to the part of the complementary strand (Sequence (A) on the template nucleic acid) exposed by formation of its structure, and the polymerase immediately proceeds extension reaction, which leads to displacement and release of the previously synthesized strand and simultaneously a new double-stranded nucleic acid is produced.

In the isothermal amplification primer method, design criteria for the first primer in a preferable aspect of the present invention are as follows. First, after a complementary strand of the template nucleic acid is synthesized by primer extension, in order to anneal the new primer to the same template nucleic acid in good efficiency, the part of Sequence (A) on the template nucleic acid needs to be single-stranded by formation of a stem-loop structure at the 5′ terminal of the synthesized complementary strand. For this purpose, the ratio of the difference (X−Y), the number of bases X of Sequence (Ac′) and the number of bases Y of a region sandwiched between Sequence (A) and Sequence (B) in the target nucleic acid sequence, to X, i.e., (X−Y)/X becomes important. However, a part which is present closer to 5′ side than Sequence (A) on the template nucleic acid and has no relation with hybridization of the primer need not to be single-stranded. In addition, in order to anneal the new primer to the template nucleic acid in good efficiency, formation of the stem-loop structure described above needs to be carried out also in good efficiency. Then, for efficient formation of the stem-loop structure, i.e., efficient hybridization of Sequence (B′) and Sequence (Bc), the distance between Sequence (B′) and Sequence (Bc), (X+Y), becomes important. Optimal temperature for a primer extension reaction is generally around 72° C. at a maximum, and at such a low temperature, it is difficult for the extension strand to undergo dissociation over a long region. Accordingly, in order to hybridize Sequence (B′) to Sequence (Bc) in good efficiency, it is considered that the number of bases between both sequences is preferably small. On the other hand, in order to make the part of Sequence (A) on the template nucleic acid single-stranded by hybridizing Sequence (B′) to Sequence (Bc), it is considered that the number of bases between Sequence (B′) and Sequence (Bc) is preferably large.

From the viewpoint described above, in the case where an intervening sequence is not present between Sequence (Ac′) and Sequence (B′) which constitute the primer, the first primer according to a preferable embodiment of the present invention is designed such that (X−Y)/X is −1.00 or more, preferably 0.00 or more, more preferably 0.05 or more, and even more preferably 0.10 or more, and in addition, 1.00 or less, preferably 0.75 or less, more preferably 0.50 or less and even more preferably 0.25 or less. Furthermore, (X+Y) is preferably 15 or more, more preferably 20 or more and even more preferably 30 or more, and in addition, preferably 50 or less, more preferably 48 or less, and even more preferably 42 or less.

In addition, in the case where an intervening sequence (the number of bases is Y′) is present between Sequence (Ac′) and Sequence (B′) which constitute the primer, the first primer according to a preferable embodiment of the present invention is designed such that {X−(Y−Y′)}/X is −1.00 or more, preferably 0.00 or more, more preferably 0.05 or more and even more preferably 0.10 or more, and in addition, 1.00 or less, preferably 0.75 or less, more preferably 0.50 or less and even more preferably 0.25 or less. Furthermore, (X+Y+Y′) is preferably 15 or more, more preferably 20 or more and even more preferably 30 or more, and in addition, preferably 100 or less, more preferably 75 or less and even more preferably 50 or less.

The first primer has a strand length enough to carry out base pair bonding with the target nucleic acid while maintaining necessary specificity under given conditions. This primer is preferably 15 to 100 nucleotides in length, and more preferably 20 to 60 nucleotides in length. In addition, the Sequence (Ac′) and Sequence (B′) which constitute the first primer are preferably 5 to 50 nucleotides in length, and more preferably 7 to 30 nucleotides in lenght, respectively. Further, if necessary, an intervening sequence which has no influence on the reaction may be inserted between Sequence (Ac′) and Sequence (B′).

The second primer which is used in the isothermal amplification primer method can be designed in the same manner as in the first primer for the complementary sequence of the target nucleic acid sequence. Accordingly, such second primer comprises Sequence (Cc′) at the 3′ end portion which hybridizes to Sequence (C) at the 3′ end portion of the complementary sequence of the target nucleic acid sequence, and comprises Sequence (D′) at 5′ side of Sequence (Cc′) which hybridizes to Sequence (Dc) complementary to Sequence (D) which is present closer to 5′ side than Sequence (C) in the complementary sequence of the target nucleic acid sequence. Preferable design criteria for the second primer are as described above for the first primer.

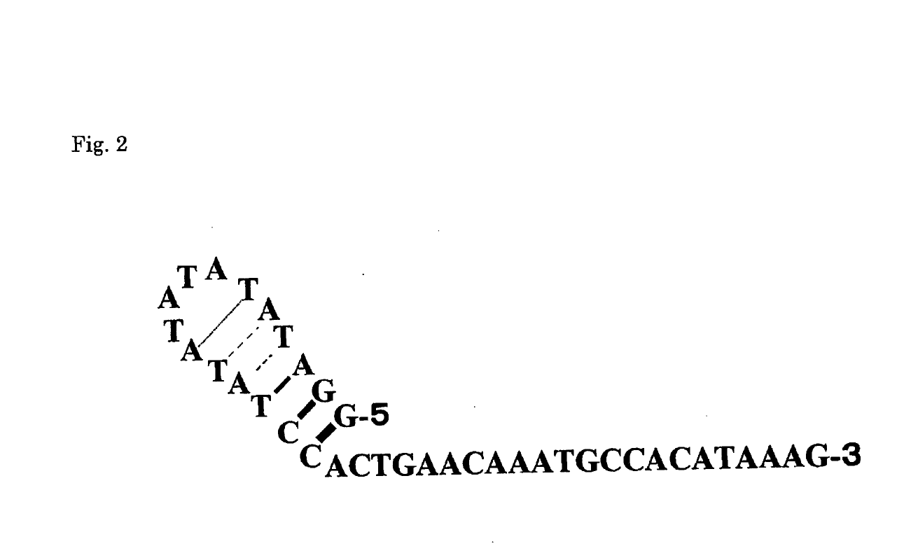

Alternatively, the second primer which is used in the isothermal amplification primer method comprises Sequence (Cc′) at the 3′ end portion which hybridizes to Sequence (C) at the 3′ end portion of the complementary sequence of the target nucleic acid sequence (the strand on the opposite side to the strand which hybridizes to the first primer), and further comprises a folded sequence (D-Dc′) at 5′ side of Sequence (Cc′) having two nucleic acid sequences which hybridize to each other on the same strand. In such isothermal amplification primer method according to the second aspect, the structure of the second primer is, for example, shown in FIG. 2, but it is not limited to the sequence or the number of nucleotides shown in FIG. 2. The Sequence (Cc′) which constitutes the second primer according to the second aspect is preferably 5 to 50 nucleotides in length, and more preferably 10 to 30 nucleotides in lenght. In addition, the folded sequence (D-Dc′) is preferably 2 to 1000 nucleotides in length, more preferably 2 to 100 nucleotides in length, even more preferably 4 to 60 nucleotides in length, and still more preferably 6 to 40 nucleotides in length, and the number of nucleotides of base pairs formed by hybridization at the inside of the folded sequence is preferably 2 to 500 bp, and more preferably 2 to 50 bp, even more preferably 2 to 30 bp and still more preferably 3 to 20 bp. The nucleotide sequence of the folded sequence (D-Dc′) may be any sequence, and it is not particularly limited, but preferably a sequence which does not hybridize to the target nucleic acid sequence. In addition, if necessary, an intervening sequence which has no influence on the reaction may be inserted between Sequence (Cc′) and the folded sequence (D-Dc′).

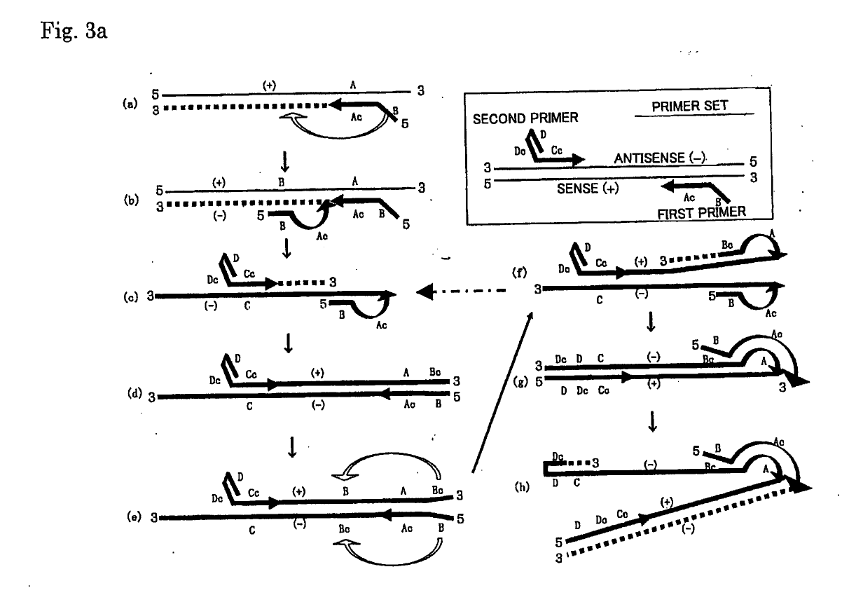

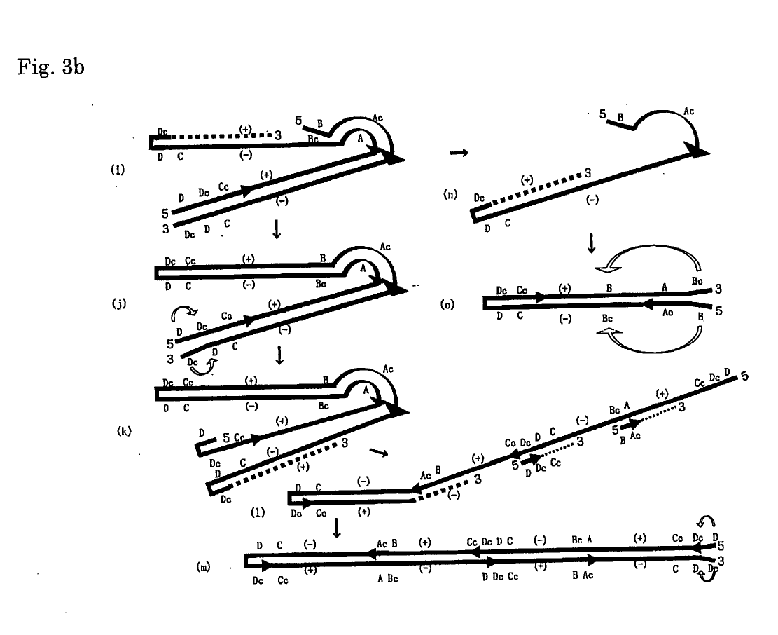

In the isothermal amplification primer method according to the second aspect, the action mechanism which is considered for the amplification reaction of a nucleic acid by the first primer and the second primer described above will be explained in reference to FIG. 3 (FIG. 3a and FIG. 3b). Here, in FIG. 3, two sequences which hybridize are exemplified as complementary sequences to each other for simple explanation, but the present invention is not limited thereto. First, the first primer hybridizes to the sense strand of the target nucleic acid, and the extension reaction of the corresponding primer takes place (FIG. 3(a)). Next, a stem-loop structure is formed on the extension strand (−),thereby a new first primer hybridizes to Sequence (A) on the sense strand of the target nucleic acid which is made single-stranded (FIG. 3(b)), and the extension reaction of the corresponding primer takes place, which leads to detachment of the previously synthesized extension strand (−). Next, the second primer hybridizes to Sequence (C) on the detached extension strand (−) (FIG. 3(c)), and the corresponding primer extension reaction takes place, which leads to synthesis of the extension strand (+) (FIG. 3(d)). A stem-loop structure is formed at the 3′ terminal of the produced extension strand (+) and 5′ terminal of the extension strand (−) (FIG. 3(e)), and extension reaction takes place at the loop tip of the extension strand (+) which is a release-type 3′ terminal, and simultaneously the extension strand (−) is detached (FIG. 3(f)). By the extension reaction at the loop tip, a hairpin type double-stranded nucleic acid is produced, wherein the extension strand (−) binds via Sequence (A) and Sequence (Bc) to the 3′ side of the extension strand (+), the first primer hybridizes to Sequence (A) and Sequence (Bc) (FIG. 3(g)), and the extension strand (−) is produced (FIGS. 3(h) and 3(i)) by its extension reaction. In addition, a release-type 3′ terminal is provided by the folded sequence which is present at the 3′ terminal of the double-stranded nucleic acid of the hairpin type (FIG. 3(h)), and by the extension reaction therefrom (FIG. 3(i)), a single-stranded nucleic acid is produced, wherein folded sequences are present at both ends, and the extension strand (+) and the extension strand (−) are alternately present via the sequence derived from the first and the second primers (FIG. 3(j)). Since the release-type 3′ terminal is provided (starting point for synthesis of the complementary strand) by the folded sequence which is present at the 3′ terminal in this single-stranded nucleic acid (FIG. 3(k)), the similar extension reaction is repeated, and the strand length is doubled (FIGS. 3(l) and 3(m)) per one extension reaction. In addition, since the release-type 3′ terminal is provided (starting point for synthesis of the complementary strand) by the folded sequence which is present at the 3′ terminal in the extension strand (−) from the first primer detached in FIG. 3(i) (FIG. 3(n)), stem-loop structures are formed at both terminals by the extension reaction therefrom, and a single-stranded nucleic acid is produced, wherein the extension strand (+) and the extension strand (−) are alternately present via the sequence derived from the primer (FIG. 3(o)). Since a starting point for synthesis of the complementary strand is sequentially provided also in this single-stranded nucleic acid by the loop formation at the 3′ terminal, the extension reaction takes place one after another therefrom. Since the sequences derived from the first primer and the second primer are contained between the extension strand (+) and the extension strand (−) in the single-stranded nucleic acid that is automatically extended as described above, it is possible that each primer hybridizes and the extension reaction takes place, thereby the sense strand and the antisense strand of the target nucleic acid are amplified remarkably.

A third primer may be used besides the first primer and the second primer in the isothermal amplification primer method. The third primer hybridizes to a target nucleic acid sequence or its complementary sequence, and has no competition with other primers for hybridization to the target nucleic acid sequence or its complementary sequence.

The “no competition” in the present invention means that hybridizing a primer to the target nucleic acid does not inhibit generation of a starting point for synthesis of the complementary strand by other primers.

Particularly, in the isothermal amplification primer method according to the second aspect, the amplification product has alternately the target nucleic acid sequence and its complementary sequence as described above in the case where the target nucleic acid is amplified by the first primer and the second primer. The folded sequence or the loop structure is present at the 3′ terminal of its amplification product, thereby the extension reaction takes place one after another from the provided starting point for synthesis of the complementary strand. The third primer can be annealed to the target sequence which is present in its single-stranded part when such amplification product becomes a partially single-stranded state. In this way, a new starting point for synthesis of the complementary strand is provided in the target nucleic acid sequence in the amplification product, and the extension reaction takes place therefrom, leading to a more rapid amplification reaction of a nucleic acid.

The third primer used in the isothermal amplification primer method is not necessarily limited to one kind, but two or more kinds of the third primer may be used at the same time in order to improve rapidity and specificity of the amplification reaction of the nucleic acid. These third primers typically comprise sequences that are different from the first primer and the second primer, but they may hybridize to a partially overlapping region as long as it does not compete with these primers. The strand length of the third primer is preferably 2 to 100 nucleotides, and more preferably 5 to 50 nucleotides and even more preferably 7 to 30 nucleotides.

The third primer used in the isothermal amplification primer method has, as its main objective, auxiliary function for more rapidly advancing amplification reaction of a nucleic acid by the first primer and the second primer. Accordingly, the third primer preferably has a Tm that is lower than the Tm of each of 3′ terminal of the first primer and the second primer. Further, the amount of addition of the third primer to the reaction solution for amplification is preferably less than the amount of addition of the first primer or the second primer.

The third primer used in the isothermal amplification primer method includes those which provide the starting point for synthesis of the complementary strand at a loop part by using a template which has a structure capable of forming the loop, as described in the pamphlet of WO 02/24902, but it is not limited thereto. That is, it may be those which provide a starting point for synthesis of the complementary strand at any site if it is within the target nucleic acid sequence.

The primer used in the isothermal amplification primer method is composed of deoxynucleotides and/or ribonucleotides. The “ribonucleotide” in the present invention (sometimes it may be abbreviated as “N”) refers to ribonucleoside triphosphate, for example, ATP, UTP, CTP, GTP and the like. Furthermore, the ribonucleotide includes derivatives thereof, for example, ribonucleotide wherein the oxygen atom of the phosphate group at the α position is substituted with a sulfur atom (α-thio-ribonucleotide) or the like.

In addition, the primer used in the isothermal amplification primer method also contains an oligonucleotide primer composed of unmodified deoxynucleotides and/or modified deoxynucleotides, an oligonucleotide primer composed of unmodified ribonucleotides and/or modified ribonucleotides, a chimera oligonucleotide primer having unmodified deoxynucleotides and/or modified deoxynucleotides and unmodified ribonucleotide and/or modified ribonucleotide and the like.

The DNA polymerase used in the isothermal amplification primer method may be those which have strand displacement activity, and any of those having room temperature properties, middle temperature properties, or heat-resistant properties can be used suitably. Further, this DNA polymerase may be either a natural one or a variant one modified by artificial variation. Furthermore, this DNA polymerase preferably has substantially no 5′→3′ exonuclease activity. Such DNA polymerase includes a variant deficient in 5′→3′ exonuclease activity of DNA polymerase derived from thermophilic bacteria of genus Bacillus such as Bacillus stearothermophilus (hereinafter, referred to as “B. st”), Bacillus caldotenax (hereinafter, referred to as “B. ca”) and the like, or Klenow fragment of Escherichia coil (E. coli)-derived DNA polymerase I and the like.

Other reagents used in the isothermal amplification primer method include, for example, a catalyst such as magnesium chloride, magnesium acetate, magnesium sulfate and the like, a substrate such as dNTP mixture and the like, and a buffer solution such as Tris hydrochloride buffer, tyrosine buffer, sodium phosphate buffer, potassium phosphate buffer and the like. Furthermore, they may include an additive such as dimethylsulfoxide (DMSO), betaine (N,N,N-trimethyl glycine) and the like, an acidic substance or a cation complex described in the pamphlet of WO 99/54455, and the like.

The primer used in the isothermal amplification primer method may comprise a recognition site for a restriction enzyme, which allows improvement of efficiency of nucleic acid amplification. That is, a nick is generated in the amplification product by the restriction enzyme corresponding to a recognition site for the restriction enzyme in the primer, which allows synthesis reaction of the complementary strand of a strand displacement type with the nick as a synthesis starting point. This method is basically based on the principle of the SDA method described as a prior art. In this method, it is necessary to design the primer such that a dNTP derivative is incorporated into the part of the complimentary strand of the reverse primer at which the nick is generated, in order to prevent cleavage of the double strand by the restriction enzyme and thus acquiring nuclease resistance.

In addition, the primer used in the isothermal amplification primer method may comprise a promoter sequence of the RNA polymerase, which allows improvement of efficiency of nucleic acid amplification. This method is basically based on the principle of the NASBA method described as a prior art.

Further, in the isothermal amplification primer method, the “outer primer” used in the LAMP method or the SDA method may be used, which allows improvement of efficiency of nucleic acid amplification. As the outer primer, there may be used a primer which can provide a starting point for synthesis of the complementary strand at the part located on the outside of the target nucleic acid sequence on the template nucleic acid.

According to the isothermal amplification primer method according to the present inventors, only in the case where the isothermal amplification primer is annealed to the target nucleic acid, causing the extension reaction from 3′ terminal side of the primer, whereby its extension product contains the desired sequence, it is possible for the 5′ terminal sequence of the primer to cause hybridization to the extension product, which then causes the similar isothermal amplification primer to be annealed, thus making continuous amplification reaction possible. Conversely, in the case where the isothermal amplification primer is annealed to a site other than the target nucleic acid by mistake and the extension reaction is caused at the 3′ terminal side of the primer, since its extension product does not contain the desired sequence, the 5′ terminal sequence of the primer cannot hybridize to the extension product, which makes it difficult for the similar isothermal amplification primer to be annealed, making continuous amplification reaction difficult and thus the desired amplification product is not obtained. Therefore, this amplification method can be said to be an amplification method with very high specificity compared to other amplification methods. Furthermore, by being an amplification method with very high specificity, this method does not necessarily need operations such as performing hybridization of the amplification product by using a DNA probe or the like, and identifying if or not the amplification product is the desired amplification product.

The isothermal amplification primer method according to the present inventors can be implemented by maintaining the temperature at which it is possible to maintain the activity of the enzyme used. In addition, in order to anneal the primer to the target nucleic acid, for example, the reaction temperature is preferably set to a temperature around or less than the melting temperature (Tm) of the primer, and furthermore, it is preferable to set a stringency level by considering the melting temperature (Tm) of the primer. Accordingly, this temperature is preferably 20° C. to 80° C., more preferably about 35° C. to about 65° C.

The test region may comprise a melting temperature regulator in order to enhance efficiency of nucleic acid amplification in the amplification reaction of a nucleic acid. The melting temperature (Tm) of a nucleic acid is generally determined by the specific nucleotide sequence of the double strand-forming portion in the nucleic acid. By adding the melting temperature regulator to the reaction solution, this melting temperature can be changed, and accordingly, it is possible to regulate the strength of double-strand formation in the nucleic acid under a constant temperature. General melting temperature regulator has an effect of decreasing the melting temperature. By adding such melting temperature regulator, it is possible to decrease the melting temperature in the double strand-forming portion between two nucleic acids. In other words, it is possible to decrease the strength of double-strand formation. Accordingly, if such melting temperature regulator is added to the reaction solution in the amplification reaction of a nucleic acid, it is possible to make the double-stranded part to single strands effectively in a GC-rich nucleic acid region which forms strong double strand or a region which forms a complicated secondary structure. Accordingly, next primer easily hybridizes to the target region after the extension reaction by the primer is terminated, which makes it possible to increase efficiency of nucleic acid amplification. The melting temperature regulator which is used in the present invention and its concentration in the reaction solution are suitably selected by those skilled in the art by considering other reaction conditions which have an influence on the hybridization condition, for example, salt concentration, the reaction temperature and the like. Accordingly, the melting temperature regulator is not particularly limited, but preferably dimethylsulfoxide (DMSO), betaine, formamide or glycerol, or any combination thereof, and more preferably dimethylsulfoxide (DMSO).

The test region may also comprise an enzyme stabilizer. Since it stabilizes the enzyme, it is possible to enhance efficiency of nucleic acid amplification. The enzyme stabilizer which is used in the present invention may be those which have been known in the related technical field such as glycerol, bovine serum albumin, saccharides and the like, and it is not particularly limited.

The test region may also comprise a reagent to intensify heat-resistance of an enzyme such as a DNA polymerase, a reverse transcription enzyme and the like. Since it stabilizes the enzyme, it is possible to enhance efficiency of nucleic acid amplification. Such reagent may be those which have been known in the related technical field, and it is not particularly limited, but it includes preferably saccharides, and more preferably monosaccharide or oligosaccharide, even more preferably trehalose, sorbitol or mannitol, or a mixture of two or more kinds thereof.

The test region may comprise a mismatch recognition protein. It allows detection of the target nucleic acid more accurately.

It has been already known that, when a base pair which cannot match partially in the double strand of DNA (mismatch) is generated, bacteria, yeast or the like have a mechanism to repair this. This repair is carried out by a protein called the “mismatch-binding protein” (also called the “mismatch recognition protein”), and various mismatch-binding proteins have been reported to be used such as MutS protein (JP No. 9-504699), MutM protein (JP-A No. 2000-300265), MutS protein bound to GFP (Green Fluorescence Protein) (the pamphlet of WO 99/06591). Furthermore, in recent years, a gene diagnosis method which detects mismatch utilizing the mismatch-binding protein has been developed (M. Gotoh et al., Genet. Anal., 14, 47-50, (1997)). As a detection method for polymorphism and mutagenesis in certain nucleotides in a nucleic acid, for example, a method of detecting a mismatch is known wherein a control nucleic acid having no variation is hybridized to a test nucleic acid which is suspicious for the presence of variation, and a mismatch recognition protein is introduced thereto.

The “mismatch” in the present invention means that a set of base pair selected from adenine (A), guanine (G), cytosine (C) and thymine (T) (uracil (U) in the case of RNA) is not normal base pair (combination of A and T, or combination of G and C). The mismatch contains not only one mismatch, but also a plurality of continuous mismatches, a mismatch generated by insertion and/or deletion of one or a plurality of bases, and a combination thereof.

Use of these mismatch-binding proteins makes it also possible to improve the specificity (accuracy) in detection of the target nucleic acid using the testing chip according to the present invention. For example, there is a case where a small amount of hetero double-stranded structure is created between the primer and the template nucleic acid in the amplification reaction of a nucleic acid. The “hetero double-stranded structure” in the present invention means a double-stranded structure which contains a non-complementary region due to the presence of one or a plurality of mismatches though it is substantially a complementary double-stranded structure. By such hetero double-stranded structure, a wrong amplification product is formed which is in principle not produced. In this way, if a mismatch-binding protein is added to the reaction system used in the amplification reaction of a nucleic acid, this mismatch-binding protein binds to a hetero double-stranded structure as described above, and inhibits subsequent amplification reaction. Accordingly, use of the mismatch-binding protein can inhibit of production of a wrong amplification product.

The mismatch-binding protein used in the present invention may be a protein which can recognize a mismatch in a double-stranded nucleic acid and bind to the mismatch site, and may be any one which is known to those skilled in the art. In addition, the mismatch-binding protein used in the present invention may be a protein which comprises an amino acid sequence wherein one or a plurality of amino acids in the amino acid sequence of a wild-type protein are displaced, deleted, added, and/or inserted (variant), as long as it can recognize a mismatch in the double-stranded nucleic acid. Such variant may be generated in the nature, but also may be constructed artificially. As a method for introducing amino acid variation into a protein, many methods are publicly known. For example, the method of W. P. Deng and J. A. Nickoloff (Anal. Biochem., 200, 81, (1992)), the method of K. L. Makamaye and F. Eckstein (Nucleic Acids Res., 14, 9679-9698, (1986)) and the like are known as a method of introducing site-specific variation. A method of using Escherichia coil XL1-Red strain (Stratagene Corp.) which lacks in the basic repairing system, a method of modifying bases chemically using sodium nitrite and the like (J.-J. Diaz et al., BioTechnique, 11, 204-211, (1991)) and the like are known as a method of introducing random variation. As such mismatch-binding protein, many proteins are known such as MutM, MutS and an analog thereof (Radman, M. et al., Annu. Rev. Genet. 20: 523-538 (1986); Radaman, M. et al., Sci. Amer., August 1988, pp. 40-46; Modrich, P., J. Biol. Chem. 264: 6597-6600 (1989); Lahue, R. S. et al., Science 245: 160-164 (1988); Jiricny, J. et al., Nucl. Acids Res. 16: 7843-7853 (1988); Su, S. S. et al., J. Biol. Chem. 263; 6829-6835 (1988); Lahue, R. S. et al., Mutat. Res. 198: 37-43 (1988); Dohet, C. et al., Mol. Gen. Gent. 206: 181-184 (1987); Jones, M. et al., Genetics 115: 605-610 (1987); Muts of Salmonella typhimurium (Lu, A. L., Genetics 118: 593-600 (1988); Haber L. T. et al., J. Bacteriol. 170: 197-202 (1988); Pang, P. P. et al., J. Bacteriol. 163: 1007-1015 (1985)); and Priebe S. D. et al., J. Bacterilo. 170: 190-196 (1988)). The mismatch-binding protein used in the present invention is preferably those derived from MutS, MutH, MutL or yeast, and more preferably MutS, MutH or MutL.

Certain mismatch-binding proteins bind to a single-stranded nucleic acid as well, and it has been known that such binding of the mismatch-binding protein to the single-stranded nucleic acid is inhibited by a single strand-binding protein. Accordingly, when a mismatch-binding protein is used in the present invention, the single strand-binding protein is preferably used in combination. In addition, some mismatch-binding proteins bind to a double-stranded nucleic acid which contains no mismatch, and it has been known that such wrong binding of the mismatch-binding protein can be inhibited by keeping the mismatch-binding protein activated using an activator in advance. Accordingly, when a mismatch-binding protein is used in the present invention, it is preferably activated in advance by an activator.

The single strand-binding protein (SSB) used to inhibit binding of the mismatch-binding protein to a single-stranded nucleic acid may be an arbitrary SSB known in the related technical field. Preferable SSB includes a single strand-binding protein derived from Escherichia coli, Drosophila and Xenopus laevis, and gene 32 protein derived from T4 bacteriophage, and an equivalent thereof derived from other species. The mismatch-binding protein used in this case includes MutS, HexA, MSH1-6, Rep3, RNase A, uracil-DNA glycosidase, T4 endonuclease VII, resolvase and the like.

The activator for activating the mismatch-binding protein may be suitably selected by those skilled in the art, and it is not particularly limited, but preferably a compound such as ATP (adenosine 5′-triphosphate), ADP (adenosine 5′-diphosphate), ATP-γ-S (adenosine 5′-O-(3-thio-triphosphate)), AMP-PNP (adenosine 5′-[β,γ-imide]-triphosphate), or nucleotides which can bind to the mismatch-binding protein. Activation of the mismatch-binding protein can be carried out by incubating the mismatch-binding protein and the activator at room temperature for several seconds to several minutes.

The test region is formed as to make a liquid sample in contact with an amplification reagent when it is in contact with the liquid sample. For example, the test region can be formed by immobilizing the amplification reagent by a water-absorbing material. In this way, the liquid sample in contact with the test region is absorbed rapidly into the inside, where it can come into contact with the amplification reagent. In addition, since the liquid sample absorbed in the water-absorbing material is retained in the water-absorbing material together with the amplification reagent, it becomes possible to maintain contact of the liquid sample with the amplification reagent in the inside of the test region over a sufficient time for performing the amplification reaction of a nucleic acid.

The water-absorbing material includes, for example, cellulose-based material such as paper material such as filter paper, a porous material, a water-absorbing polymer and the like. The water-absorbing polymer includes, for example, a polymer made of starch or its derivative, a polymer made of cellulose or its derivative, a polymer made of polyacrylate or its derivative, polyvinylalcohol, starch/acrylic copolymer, polyethyleneoxide, acrylate/vinyl alcohol copolymer, isobutylene/maleate copolymer, starch/acrylate copolymer, crosslinked carboxymethyl cellulose and the like. As the water-absorbing material, one kind of the material may be used alone, or a mixture of two or more kinds of the materials may also be used.

Immobilization of the amplification reagent to the water-absorbing material may be carried out by a standard method well-known in the related technical field, and its specific procedure is not particularly limited. Such method includes, for example, a method wherein, after a water-absorbing material is prepared, the amplification reagent solution is added thereto to swell this and then it is dried. Another method includes a method wherein, after an amplification reagent is bound to particles such as beads, the obtained particles are mixed with a solution used to prepare the water-absorbing material and a water-absorbing material is prepared using this mixed solution. Furthermore, it includes another method wherein an amplification reagent is covalently bonded to the water-absorbing material. For example, a method of binding an oligonucleotide such as a primer and the like to the water-absorbing material includes a method of using a binding reaction with biotin and avidin, a method of using a reactive group such as an amino group or a hydroxyl group in a nucleotide molecule (See, for example, Ichiro Senhata, et al., “Experiment and Application: Affinity chromatography”, pp. 16-106, published by Kodansha Scientific Ltd.). In addition, a method of binding an enzyme to a water-absorbing material includes a standard method used in the field of immobilized enzyme (See, for example, “Immobilized Enzyme”, edited by Ichiro Senhata, (1975), published by Kodansha Ltd.). The amplification reagent is preferably immobilized so as to be retained in the inside of the detection region without outflow from the detection region after it has come into contact with the liquid sample.

The water-absorbing material may be placed in the inside or on the surface of the support by a standard method well-known in the related technical field, and its specific procedure is not particularly limited. Such method includes, for example, a method of incorporating a water-absorbing material into a location where a test region is formed when molding a material to become a support. It includes another method wherein the water-absorbing material is mixed in the form of particles in a material to be a support, and a support is molded so that the part which contains these particles is placed in a location where the test region is to be formed. Furthermore, it includes another method wherein a support is constructed, the water-absorbing material is applied to the location where the test region is to be formed, and this is dried. Furthermore, it includes another method wherein a support is constructed, the water-absorbing material is multi-layered at the location where the test region is to be formed, and then this is coated with a water-permeable material. Such coating material may be those which permeate water, and it is not particularly limited. In addition, the form of the coating material may be in various forms such as film and mesh. As the coating material, may be used, for example, a material having a unidirectional water permeability, namely, a material which permeates water from the outside toward the test region but does not permeate water from the test region toward the outside. Alternatively, as the coating material, may be used a material which permeates water but does not permeate erythrocyte or hemoglobin in the blood (See, for example, JP-A No. 6-86696), which allows easily visual detection of the target nucleic acid in the case where the blood is used as a liquid sample.

The testing chip according to the present invention further comprises a sample introduction part provided on the support. This sample introduction part is provided on the surface of the support and placed such that the introduced liquid sample can come into contact with the test region. For example, in the case where the test region is located on the surface of the support, it is possible to introduce the liquid sample directly to the test region, and accordingly, the test region itself can be made a sample introduction part. Alternatively, a sample introduction part can be also placed at a different location from the test region. In this case, if necessary, a liquid transporting means is provided to transport the liquid sample from the sample introduction part to the test region. Such liquid transporting means includes, for example, forming the region from a sample introduction part in the support to the test region by using a material which can transport the liquid sample by capillary phenomenon (for example, a filter paper) and the like.

The target nucleic acid amplified by using the testing chip according to the present invention may be detected by a general method, for example, a method using a specific probe to which a detectable label is attached and the like, but it is also possible to constitute the testing chip so as to generate a signal based on the presence of the amplification product. Therefore, according to a preferable embodiment of the present 4invention, the testing chip according to the present invention further comprises a signal generation means which generates a signal due to the amplification product. In addition, in this case, the testing chip according to the present invention is preferably permeable to the signal from the nucleic acid amplification product.

As the signal generation means, those known to those skilled in the art can be used, and they are not particularly limited, but for example, intercalators such as ethidium bromide and SYBR green 1 (Molecular Probe) can be used. Since these intercalators bind to a double-stranded DNA, its fluorescence intensity is positively proportional to the concentration of the double-stranded DNA. Therefore, if the fluorescence by an intercalator is strong, it shows that the amplification product is present in a high concentration, thereby the target nucleic acid is detected. Accordingly, by incorporating such intercalator into the amplification reagent in advance, it is possible to generate a signal based on the amplification product. In addition, as the signal generation means, Fluorescence Resonance Energy Transfer (FRET) and the like may be utilized. FRET is generated only in the case where two probes approach to each other and hybridize to the amplification product, and it is not generated in the case where a specific DNA capable of mutual hybridization is not present adjacent to hybridization probes. Accordingly, it is good to incorporate two probes, which can hybridize specifically to each of two adjacent regions of the target nucleic acid in advance, into the amplification reagent. In addition, in the process of nucleic acid amplification, pyrophosphate ion is released from the substrate (dNTPs), which binds to magnesium ion in the composition of the amplification reagent to produce magnesium pyrophosphate, thereby the reaction solution becomes clouded, whereby the presence or absence of the amplification product can be visually detected. Further, detection can be also carried out by inserting an intercalating agent into the amplification product, flowing electric current by using this in the amplification product, and reading out difference of the current or voltage. Furthermore, the primers may be bound in advance to a carrier such as beads or gold colloidal particles. In this case, if the target nucleic acid is amplified, the carrier is aggregated, so identifying this by visual inspection allows detection of the amplification product. According to a preferable embodiment of the present invention, the signal is detectable by visual inspection as the signal generation means.

The testing chip according to the present invention, if necessary, can be made to further comprise a part for immobilizing a nucleic acid extraction reagent wherein the nucleic acid extraction reagent is immobilized to extract a nucleic acid from a liquid sample. This part for immobilizing the nucleic acid extraction reagent can be placed between the sample introduction part and the test region in the inside or on the surface of the support. In this way, even in the case where the target nucleic acid is not present in an exposed state in the liquid sample, for example, in the case where the target nucleic acid is present in the cell in the liquid sample, the target nucleic acid can be easily detected. The reagent contained in this extraction reagent is not particularly limited, and may be those with which are performed various methods for extraction of a nucleic acid known to those skilled in the art. The composition of the extraction reagent can be suitably determined by those skilled in the art depending on the method for extraction of the nucleic acid to be used.

As the method for extraction of a nucleic acid, for example, are known alkali extraction, phenol extraction, chaotropic reagent extraction, a method using a surfactant, chromatographic purification (WO 95/01359) and ultracentrifugation (Maniatis, et al., (1982), Molecular Cloning: A Laboratory Manual, Cold Spring Harbor Laboratory, Cold Spring Harbor, N.Y.). In addition, a method is also known for extracting a nucleic acid by lysis of protein in a sample using a non-specific proteolytic enzyme such as proteinase K, protease, subtilisin and the like. In the case where a proteolytic enzyme is used, it is preferred that the proteolytic enzyme is immobilized by a covalent bond to the part for immobilizing a nucleic acid extraction reagent, or the part for immobilizing the nucleic acid extraction reagent is spaced enough from the test region so that the nucleic acid extraction reagent is not mixed with the amplification reagent. As the surfactant, any of those cationic, anionic, amphoteric and non-ionic may be used. Such surfactant includes, for example, cetyl trimethyl ammonium bromide (CTAB), sodium dodecyl sulfate (SDS), sodium N-lauroylsarcosine, CHAPS (3-[(3-cholamidopropyl)-dimethylammonio]-1-propanesulfonic acid), polyoxyethylene sorbitan monolaurate (Tween 20) and the like, but it is not limited thereto. In addition, it is also possible to dissolve the cells using lysozyme.

According to a preferable embodiment of the present invention, the nucleic acid extraction reagent includes a reagent for alkali extraction, a reagent for protease reaction or a surfactant, or a combination thereof, and more preferably a reagent for alkali extraction, further preferably sodium hydroxide.

The reagent for alkali extraction may also comprise the surfactant described above. The concentration of the surfactant in the reagent for alkali extraction is not particularly limited, but preferably 0.005 to 5% (w/v), and more preferably 0.01 to 2% (w/v).

As the method for extraction of a nucleic acid, may be used a method of extracting a nucleic acid by decomposition or denaturation of proteins and other adulterated substances in the sample using a protein-denaturing agent, and this method is particularly effective for extracting RNA. The protein-denaturing agent may be those which can solubilize a protein, and it is not particularly limited, but includes, for example, guanidine salts such as guanidine hydrochloride, guanidine thiocyanate, guanidine carbonate and the like, a chaotropic substance comprising urea and the like, and the like. It is particularly preferably guanidine hydrochloride, guanidine thiocyanate and the like. By using a protein-denaturing agent, it is possible to suppress in good efficiency the action of RNase which is likely mixed in a bio-sample. In addition, a chelating agent which can suppress the action of a nuclease such as sodium citrate, EDTA and the like, or a reducing agent such as dithiothreitol (DTT), β mercaptoethanol and the like may be used.

In the case where a part for immobilizing the nucleic acid extraction reagent is provided in the testing chip according to the present invention, the amplification reagent is preferably constituted such that it has a suitable pH for the amplification reaction when it is mixed with a liquid sample by way of the nucleic acid extraction reagent. For example, in the case where a reagent for alkali extraction is used as the nucleic acid extraction reagent, when the pH of the liquid sample passing through this reagent turns out to be too high for the amplification reaction, the pH of the amplification reagent when dissolved in water is preferably set to be low in advance. A suitable range of pH in the amplification reaction is approximately in a range of 5 to 12, preferably in a range of 7 to 10.

According to another embodiment of the present invention, the testing chip according to the present invention, if necessary, may further comprise a part for immobilizing a pH regulating reagent wherein the pH regulating reagent is immobilized in order to make the pH of the reaction solution for nucleic acid amplification formed in the test region suitable for the amplification reaction by the amplification reagent. This part for immobilizing the pH regulating reagent can be placed between the part for immobilizing the nucleic acid extraction reagent and the test region in the inside or on the surface of the support.

An acid which can be used as the pH regulating reagent includes mineral acids such as hydrochloric acid, sulfuric acid, nitric acid, phosphoric acid and the like, carboxylic acids such as acetic acid, citric acid, phthalic acid, fumaric acid, maleic acid and the like, organic sulfonic acids such as methanesulfonic acid, p-toluenesulfonic acid and the like, preferably mineral acids, and further preferably hydrochloric acid. In addition, an alkali which can be used as the pH regulating reagent includes typically sodium hydroxide.

Immobilization of the reagent in the part for immobilizing the nucleic acid extraction reagent and in the part for immobilizing the pH regulating reagent can be carried out by the method described above for the amplification reagent. For example, the part for immobilizing the nucleic acid extraction reagent and the part for immobilizing the pH regulating reagent can be formed by infiltrating each reagent solution into a porous carrier, drying it, and then placing the obtained porous carrier at a predetermined location on the testing chip.

The testing chip according to the present invention may further comprise a reagent for adsorbing foreign substances in order to adsorb foreign substances that hinder the amplification reaction of a nucleic acid, which can be placed in the test region, or in the part for immobilizing the nucleic acid extraction reagent or the part for immobilizing the pH regulating reagent. Alternatively, the part for immobilizing the reagent for adsorbing foreign substances wherein the reagent for adsorbing foreign substances is immobilized can be provided somewhere between the sample introduction part and the test region. Such reagent for adsorbing foreign substances includes typically active carbon.

The form of the testing chip according to the present invention is not particularly limited, but preferably it is in the form of a test specimen, a sheet or a tape. In addition, the testing chip according to the present invention may comprise a plurality of test regions for amplifying a plurality of kinds of target nucleic acids individually. In this case, if necessary, the liquid transporting means described above is provided in order to transport a liquid sample from the sample introduction part to all of the test regions. In addition, it is preferred that the plurality of test regions are placed in parallel with the sample introduction part, or such reagent is kept as immobilized to each of the test regions by covalent bond, to avoid mixup of the reagents (for example, the primer) that are specific to each of the target nucleic acids contained in each of the test regions.

The testing chip according to the present invention is used to detect a target nucleic acid from a liquid sample. Accordingly, according to another aspect of the present invention, a method is provided for detecting the target nucleic acid from a liquid sample using the testing chip according to the present invention, which comprises steps of,
(a) bringing a liquid sample into contact with a sample introduction part on the testing chip;
(b) incubating the testing chip under conditions suitable for an amplification reaction of a nucleic acid by an amplification reagent contained in a test region; and
(c) detecting a signal from a nucleic acid amplification product of the amplification reaction of the nucleic acid.

The liquid sample may be those which are suspicious for containing the target nucleic acid and it is not particularly limited, but includes, for example, a sample derived from a living organism, a processed food, waste water, drinking water and the like. In addition, the living organism may be any of animals, plants and microorganisms. Further, the animal is preferably mammal, and more preferably human. The sample from animals includes blood, feces, phlegm, mucous membrane (oral cavity, nasal cavity and the like), mucus, serum, urine, saliva, lacrimal fluid, biopsy sample, histological tissue sample, tissue culture and the like. In addition, the plants include agricultural crops, foliage plants, natural edible plants and the like. Such liquid sample may be any one containing water. In addition, the sample which contains little water may be made a liquid sample by adding water.

The target nucleic acid may be those from which useful information is obtained by detection and it is not particularly limited, but includes, for example, a wild-type gene, a variant-type gene or those containing a nucleic acid sequence specific to a pathogen. The pathogen includes, for example, virus, bacterium, fungus and the like. For example, in the case where a wild-type gene is the target nucleic acid, a disease due to deficiency of its gene is detected by non-detection of the gene. In addition, in the case where a variant-type gene is the target nucleic acid, a disease due to the gene variation is detected by detection of the gene. Further, in the case where a nucleic acid having a sequence specific to a pathogen is the target nucleic acid, infection by its pathogen is detected by detecting the nucleic acid.

Each step in the method for detecting a nucleic acid according to the present invention can be easily implemented depending on the constitution of the testing chip according to the present invention, for example, depending on the amplification method for the nucleic acid used in the corresponding testing chip. In addition, detection of a signal from the nucleic acid amplification product can be suitably carried out by those skilled in the art using a general method such as a method of using a specific probe attached with a detectable label. Further, in the case where a signal generation means is present in advance in the testing chip, signal detection can be simply carried out using this.

As a means of utilizing effectively the results of the method for detecting a nucleic acid according to the present invention, it is also possible to input the signal detected in step (c) or results obtained by the signal into a computer for gene analysis, and to output the results of analysis by the computer. Therefore, according to the present invention, a method for gene analysis is provided which comprises, after steps of (a) to (c):
(d) inputting the detected signal into a computer for gene analysis,
(e) comparing the signal with information available by the computer in the computer to characterize the signal and/or to search for the information related to the signal; and
(f) outputting the characteristics of the signal and/or the information related to the signal from the computer. The input into the computer in step (e) and the output from the computer in step (f) are preferably carried out via a communication network such as the internet.

According to the method for gene analysis according to the present invention, for example, it becomes possible to obtain more detailed information by connecting a device for signal detection and a communication device, transmitting the obtained signal to a gene analysis center and the like which performs more detailed analysis, and receiving detailed results of the analysis and related information. As the communication device, suitably used are a portable terminal such as a personal computer, a portable telephone and the like, which can perform transmission and reception of information via a communication network such as the internet and the like. Alternatively, it is also possible to use a device which can perform the method for gene analysis according to the present invention by mounting the testing chip according to the present invention. Such device includes, for example, a portable terminal such as a portable telephone provided with a heat insulation means, a moisturizing means and the like in order to maintain suitable conditions for the amplification reaction of a nucleic acid, and the like, and furthermore, it may further comprise a signal detection means.

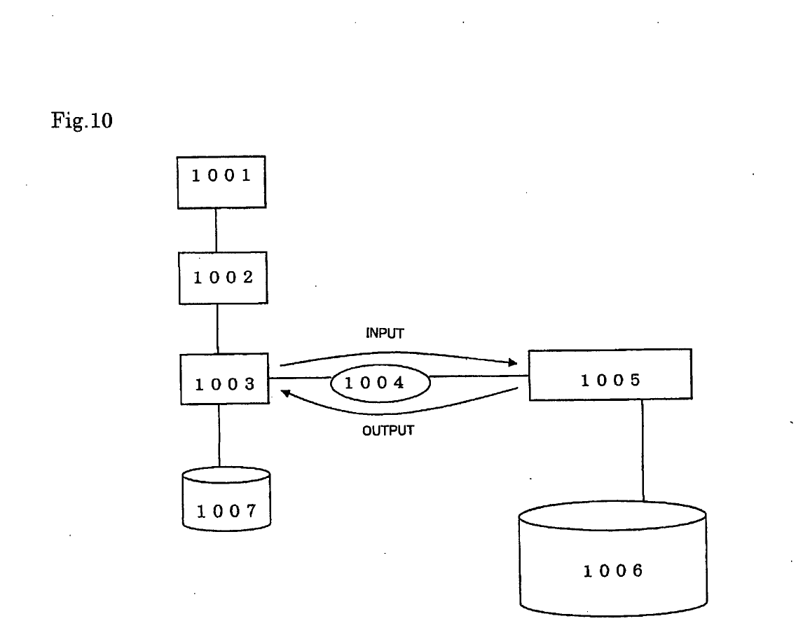

A conceptual diagram of the method for gene analysis according to a preferable embodiment of the present invention is shown in FIG. 10. The amplification reaction of a target nucleic acid is carried out in the testing chip 1001 according to the present invention, and then a signal due to the amplification product is detected by a signal detection device 1002. The detected signal is inputted into a computer for gene analysis 1005 via the internet 1004 by a portable terminal 1003. In the computer for gene analysis 1005, the inputted signal is compared with the information given by the presence or absence of the target nucleic acid stored in an information storage device 1006, from which search for information related to characterization of the signal and/or information related to the signal is carried out. Next, the information related to the characteristics of the signal and/or the signal are outputted from the computer for gene analysis 1005 by the portable terminal 1003 via the internet 1004. The outputted information is stored in an information storage device 1007 by the portable terminal 1003.

The method for gene analysis according to the present invention can be, for example, a method for detection of a disease or disorder, and further a method for acquisition of information related to the disease or disorder if the target nucleic acid shows the disease or disorder by its presence or absence. In this case, characteristics of outputted signal includes the name of the disease or disorder shown by the signal, the name of the gene having the target nucleic acid or the like, and the outputted related information includes explanation for the disease or disorder, treatment methods and effective therapeutic medication for the disease or disorder, a method for further precise diagnosis or the like. Particularly, since the computer for gene analysis enables a complicated analysis, in the case where a plurality of involved in the disease or disorder concerned genes are present, more accurate analysis results can be obtained by carrying out the method for detecting a nucleic acid for each of the genes and transmitting whole of the results (signal).

Particularly, since the method for detecting a nucleic acid according to the present invention can be performed by a subject by oneself, when a gene analysis is performed also by the subject by oneself using a communication device, leak of gene information is prevented. Furthermore, it is possible to keep and/or manage complicated gene information by managing individual information using a portable terminal and the like possessed by the individual. In addition, based on its gene information, a hospital or a store can be selected corresponding to the purpose of the individual.

### EXAMPLES

The present invention will be explained in detail below by Examples, but the scope of the present invention is not limited to these examples.

### Example 1

### Detection of a Target Nucleic Acid Sequence in Human STS DYS237 Gene

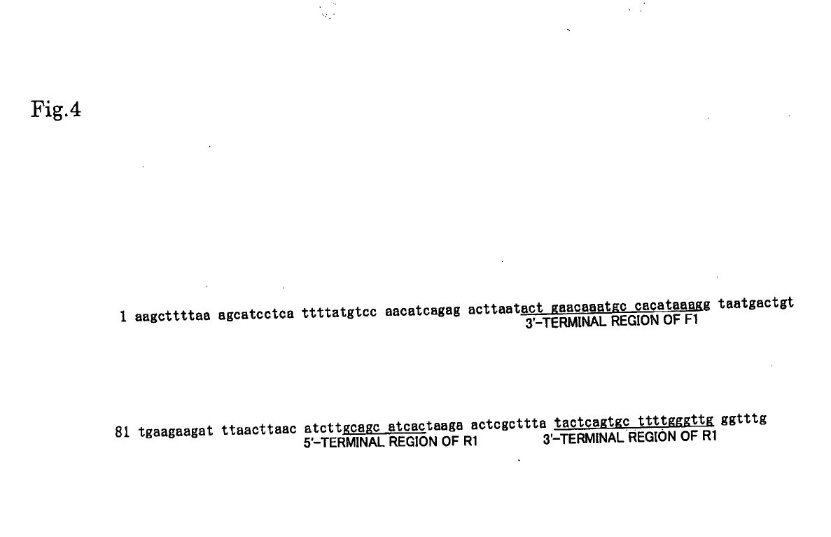

In this example, detection of human STS DYS237 gene contained in a sample solution was carried out. As the primers, a primer pair which has the sequences described below was used. In addition, the relative location of each primer region of the template is as shown in FIG. 4 (Sequence No. 9). The forward primer F1 was designed such that the sequence on the 3′ terminal side (22 mer: the underlined part) was annealed to the template, and the sequence on the 5′ terminal side (16 mer: other than the underlined part) was folded in its region to have the structure shown in FIG. 2. The reverse primer R1 was designed such that the sequence on the 3′ terminal side (20 mer: the underlined part) was annealed to the template, and after the extension reaction, the sequence on the 5′ terminal side (10 mer: other than the underlined part) was hybridized to a region which starts at 16 bases downstream of the 3′ terminal residue of the primer on the extension strand by its primer. Primer pair:

(Sequence No. 1)

F1: GGATATATATATATCCACTGAACAAATGCCACATAAAG;
and

(Sequence No. 2)

R1: GCAGCATCACCAACCCAAAAGCACTGAGTA.

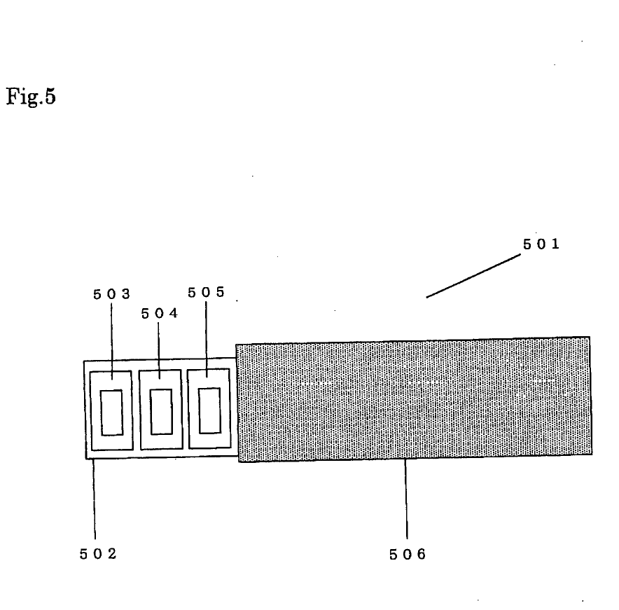

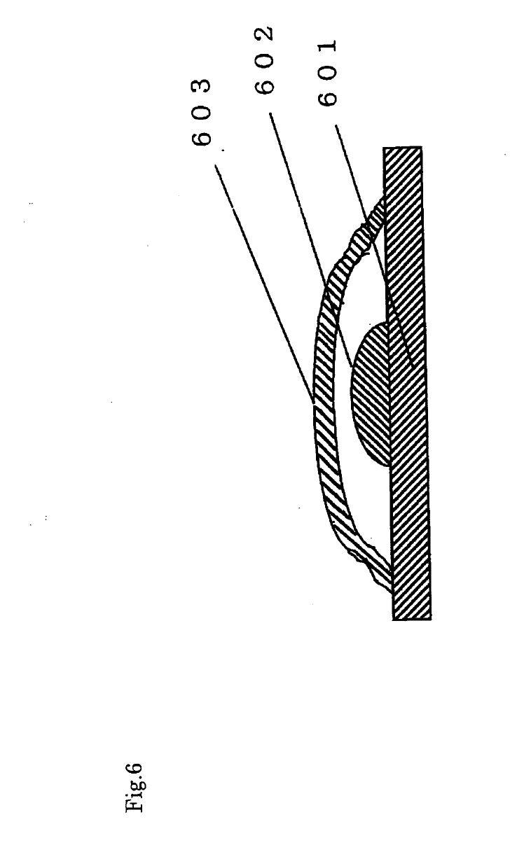

Next, an amplification reaction solution having the following composition was prepared: Tris-HCl (20 mM, pH 8.8), KCl (10 mM), (NH₄)₂SO₄ (10 mM), MgSO₄ (8 mM), DMSO (3%), Triton X-1000 (1%), dNTP (1.4 mM), 640 U/ml Bst DNA polymerase (NEW ENGLAND BioLabs), 2000 nM each of the primer pair, and ethidium bromide (0.5 μg/ml). To a polyacrylate-based high water-absorbing resin (trade name: RHEOGIC; manufactured by Nihonjunyaku, Co., Ltd.), the amplification reaction solution was added in 50 folds (by weight) thereof. After the resultant mixture was sufficiently swollen, it was dried at 4° C. to powder. The obtained powder was dispersed in Test region 1 (503) in a testing chip (501) which comprises a filter paper having 0.5 mm thickness shown in FIG. 5. After retaining the powder on the surface of the filter paper by applying pressure to the Test region 1 (503) from the top, the test region was coated with a mesh (603) as shown in FIG. 6. Test region 2 (504) was constructed as a negative control in the same manner using an amplification reaction solution without a primer.

Next, a sample solution was prepared by dissolving 100 ng of Human Genomic DNA (manufactured by Clontech Laboratories, Inc.) in 0.1 ml of water. This sample solution was infiltrated into the testing chip described above, which was incubated at 60° C. for 1 hour under the conditions where the testing chip was not dried up.

Ultraviolet ray was irradiated to the obtained testing chip to observe fluorescence intensity in each of the test regions. As a result, strong fluorescence was found in Test region 1 (503), whereas no fluorescence was found in Test region 2 (504) which is the negative control. From this result, it was shown that, by using the testing chip described above, it is possible to amplify and detect the target nucleic acid present in the sample.

### Example 2

### Detection of Single Nucleotide Polymorphism in the Human Acetaldehyde Dehydrogenase Gene

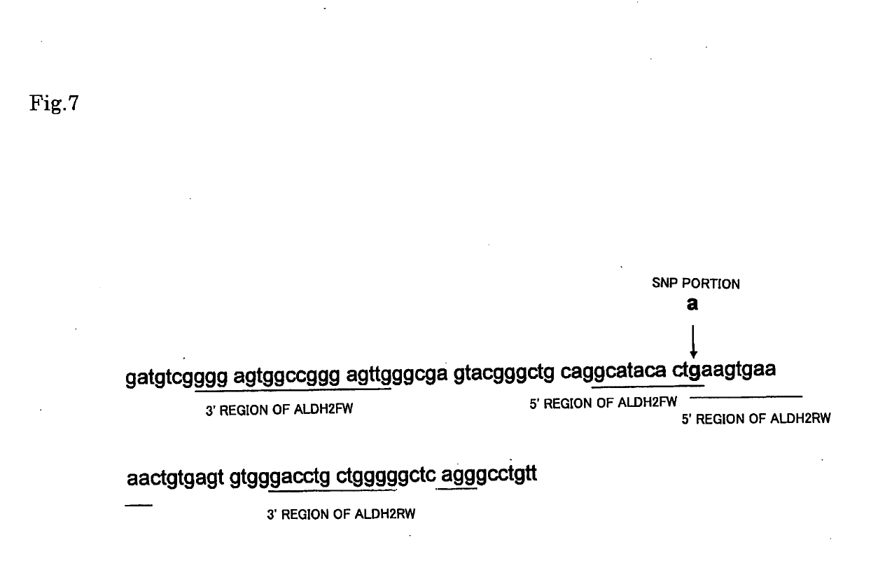

In this example, detection was carried out for single nucleotide polymorphism in the human acetaldehyde dehydrogenase gene contained in a sample solution. The single nucleotide polymorphism (SNP) present in the 12th exon of the gene was selected as the single nucleotide polymorphism for detection. The sequence around this single nucleotide polymorphism (Sequence No. 10) is shown in FIG. 7 together with the location of the region used in the design of the primer. As the primer, 2 sets of primer pairs having the sequences described below were used. The forward primers ALDH2FW and ALDH2FM were designed such that the sequence on the 3′ terminal side (18 mer: the underlined part) was annealed to the template, and after the extension reaction, the sequence on the 5′ terminal side (10 mer: other than the underlined part) was hybridized to a region which starts at 19 bases downstream of the 3′ terminal residue of the primer on the extension strand by its primer. The reverse primers ALDH2RW and ALDH2RM were designed such that the sequence on the 3′ terminal side (19 mer: the underlined part) was annealed to the template, and after the extension reaction, the sequence on the 5′ terminal side (10 mer: other than the underlined part) was hybridized to a region which starts at 13 bases downstream of the 3′ terminal residue of the primer on the extension strand by its primer. In addition, these primers were designed such that each 5′ terminal residue corresponded to nucleotide residues related to the variation.

Primer Pair for Detection of the Wild-Type DNA:

(Sequence No. 3)

ALDH2FW: CAGTGTATGCGGGAGTGGCCGGGAGTTG;
and

(Sequence No. 4)

ALDH2RW: GAAGTGAAAACCTGAGCCCCCAGCAGGTC.

Primer Pair for Detection of the Variant-Type DNA:

(Sequence No. 5)

ALDH2FM: TAGTGTATGCGGGAGTGGCCGGGAGTTG;
and

(Sequence No. 6)

ALDH2RM: AAAGTGAAAACCTGAGCCCCCAGCAGGTC.

Next, an amplification reaction solution having the following composition was prepared: Tris-HCl (20 mM, pH 8.8), KCl (10 mM), (NH₄)₂SO₄ (10 mM), MgSO₄ (2 mM), DMSO (8%), Triton X-1000 (1%), dNTP (0.2 mM), 320 U/ml Bst DNA polymerase (NEW ENGLAND BioLabs), 1200 nM each of the primer pair, MutS (10 μg/ml), and ethidium bromide (0.5 μg/ml). To a polyacrylate-based high water-absorbing resin (trade name: RHEOGIC; manufactured by Nihonjunyaku, Co., Ltd.), the amplification reaction solution was added in 50 folds (by weight) thereof. After the resultant mixture was sufficiently swollen, it was dried at 4° C. to powder. The obtained powder was dispersed in the test region in a testing chip which comprises a filter paper having 0.5 mm thickness (FIG. 5). After retaining the powder on the surface of the filter paper by applying pressure to this test region from the top, the test region was coated with a mesh (603) as shown in FIG. 6. Test region 1 (503) was constructed as a negative control in the same manner using an amplification reaction solution without a primer. Test region 2 (504) was constructed using an amplification reaction solution containing the primer pair for detection of the wild-type DNA, and Test region 3 (505) was constructed using an amplification reaction solution containing the primer pair for detection of the variant-type DNA.

Next, 3 kinds of genome DNAs, namely, a wild type specimen which has the gene type of G/G in the SNP, a variant type specimen which has the gene type of A/A, and a hetero type specimen which has the gene type of G/A, were prepared. Sample solutions were prepared by dissolving 10 ng each of DNAs in 0.1 ml of water. These sample solutions were infiltrated into the testing chip described above, which was incubated at 60° C. for 1 hour under the conditions where this testing chip was not dried up. For the obtained testing chip, the results obtained by observing the fluorescence intensity in each of the test regions on the UV illuminator are shown in Table 1 below.

TABLE 1

| | Test region 1 | Test region 2 | Test region 3 |
|---|---|---|---|
| Wild-type specimen: | − | +++ | − |
| G/G | | | |
| Variant-type specimen: | − | − | ++ |
| A/A | | | |
| Hetero type specimen: | − | + | + |
| G/A | | | |

From these results, it was shown that, by using the testing chip described above, it is possible to amplify and detect specifically any of the wild-type gene or the variant-type gene present in the sample as the target nucleic acid, from which it is possible to detect single nucleotide polymorphism.

### Example 3

### Detection of Type B Hepatitis Virus

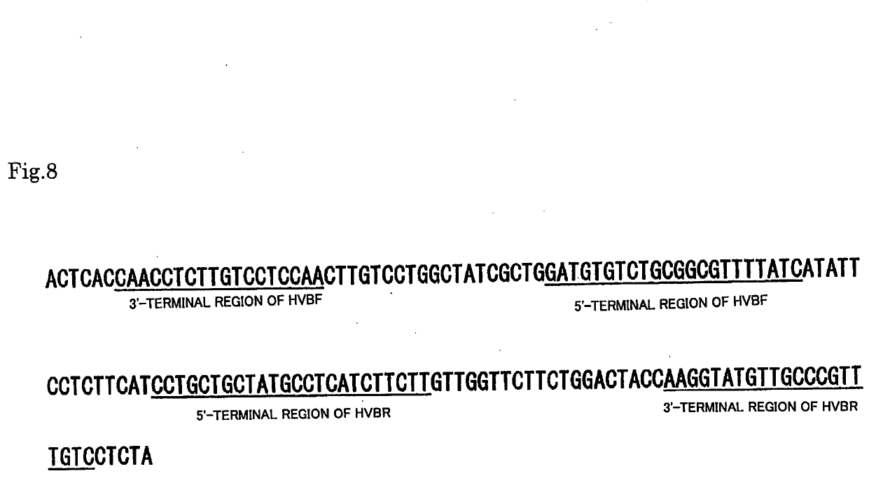

In this example, detection of Type B hepatitis virus contained in a sample solution was carried out. A primer pair having the sequences described below was used as the primer pair meant to amplify the target nucleic acid sequence specific to Type B hepatitis virus. In addition, the relative location of each primer region of the template is as shown in FIG. 8 (Sequence No. 11). The forward primer HBVF was designed such that the sequence on the 3′ terminal side (18 mer: the underlined part) was annealed to the template, and after the extension reaction, the sequence on the 5′ terminal side (22 mer: inside the bracket) was hybridized to a region which starts at 20 bases downstream of the 3′ terminal residue of the primer on the extension strand by its primer. The reverse primer HBVR was designed such that the sequence on the 3′ terminal side (21 mer: the underlined part) was annealed to the template, and after the extension reaction, the sequence on the 5′ terminal side (24 mer: inside the bracket) was hybridized to a region which starts at 21 bases downstream of the 3′ terminal residue of the primer on the extension strand by its primer.

Primer Pair:

HBVF:

(Sequence No. 7)

[GATAAAACGCCGCAGACACATC]CTTCCAACCTCTTGTCCTCCAA;
and
HBVR:

(Sequence No. 8)

[CCTGCTGCTATGCCTCATCTTCTT]TGACAAACGGGCAACATACCTT.

Next, a water-absorbing polymer composed of a mixture of starch+polyacrylic acid/polyvinyl alcohol for immobilizing the amplification reagent was prepared according to the method of Miyake et al. (The report of “Research on the development of general-use water-absorbing material (1)”, (1998), North Eastern Industrial Research Center of Shiga Prefecture). First, a mixture containing starch and acrylic acid in a weight ratio of 20:80 was dissolved in water in a concentration of 10% by weight. To 50 g of the obtained aqueous solution, 0.25 g of potassium peroxodisulfate as a polymerization initiator and 0.01 g of N,N-methylenebisacrylamide as a cross-linking agent were added. The obtained solution was heated from room temperature to 80° C. over about 40 minutes with stirring in a flask filled with an inert gas, and then stirring was continued at 80° C. for 2 hours. After neutralizing the obtained viscous solution with a 1 N aqueous solution of sodium hydroxide, PVA gel (10% by weight) was added thereto in an amount of 2 folds (weight ratio), and the mixture was stirred for 2 hours with heating.

To the obtained polymer solution, each of amplification reagents shown below was added to obtain the final concentration: Tris-HCl (20 mM, pH 8.8), KCl (10 mM), (NH₄)₂SO₄ (10 mM), MgSO₄ (2 mM), DMSO (8%), Triton X-1000 (1%), dNTP (0.2 mM), 800 U/ml Bst DNA polymerase (NEW ENGLAND BioLabs), 2000 nM of DNA polymerase aptamer, 2000 nM each of the primer pair, and SYBR green (0.5 μg/ml).

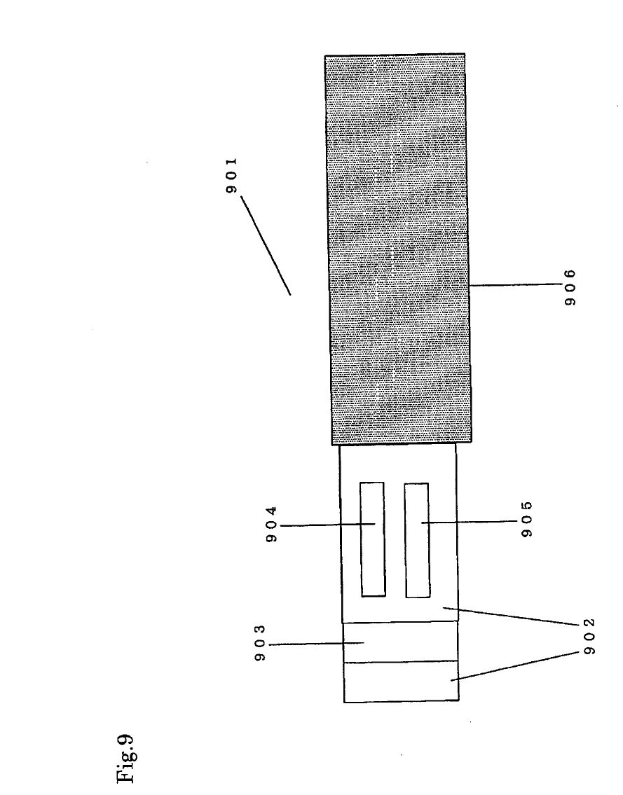

10 μl of this polymer solution containing the amplification reagent was applied to Test region 1 (904) in a testing chip which comprises a filter paper (FIG. 9), which was dried naturally. Test region 2 (905) was constructed as a negative control in the same manner using a polymer solution containing the amplification reagent except the primers.

A part for immobilizing the nucleic acid extraction reagent (903) on the testing chip (FIG. 9) was prepared by infiltrating a 0.2 N aqueous solution of sodium hydroxide into the filter paper and drying it.

Blood collected from Type B hepatitis virus (HBV)-infected persons and HBV-uninfected persons was used to prepare a sample solution. This sample solution was infiltrated into the end on the opposite side of the test region in the testing chip described above across the part for immobilizing a cell lysis agent, and this testing chip was incubated at 60° C. for 1 hour under the conditions where this testing chip was not dried up.

The results obtained by observing the fluorescence intensity in each of the test regions after UV irradiation on the obtained testing chip are shown in Table 2 below.

TABLE 2

| | Test region 1 | Test region 2 |
|---|---|---|
| HBV-infected person 1 | +++ | − |
| HBV-infected person 2 | ++++ | − |
| HBV-infected person 3 | + | − |
| HBV-infected person 4 | − | − |
| HBV-uninfected person 1 | − | − |
| HBV-uninfected person 2 | − | − |
| HBV-uninfected person 3 | − | − |

Further, even in the case without UV irradiation, it was possible to observe color development proportional to the strength of the fluorescence intensity shown in Table 2. From these results, it was shown that, by using the testing chip described above, it is possible to detect Type B hepatitis virus present in the blood sample collected from human.

1. A testing chip for detecting a target nucleic acid from a liquid sample which at least comprises a support, a sample introduction part provided on the support, and a test region on which an amplification reagent for amplifying the target nucleic acid is immobilized, wherein the test region is located in the inside or on the surface of the support.

2. The testing chip according to claim 1, wherein the amplification reagent allows an amplification of the target nucleic acid under a definite temperature.

3. The testing chip according to claim 2, wherein a first primer contained in the amplification reagent comprises Sequence (Ac′ ) at the 3′ end portion which hybridizes to Sequence (A) at the 3′ end portion of the target nucleic acid sequence, and Sequence (B′ ) at 5′ side of the Sequence (Ac′ ) which hybridizes to Sequence (Bc) complementary to Sequence (B) present closer to the 5′ side than the Sequence (A) in the target nucleic acid sequence.

4. The testing chip according to claim 3, wherein in the case where an intervening sequence is not present between the Sequence (Ac′ ) and the Sequence (B′ ) in the first primer, (X−Y)/X is in a range of −1.00 to 1.00 wherein X represents the number of bases of the Sequence (Ac′ ) and Y represents the number of bases of a region sandwiched between the Sequence (A) and the Sequence (B) in the target nucleic acid sequence, and in the case where an intervening sequence is present between the Sequence (Ac′ ) and the Sequence (B′ ) in the primer, {X−(Y−Y′ )}/X is in a range of −1.00 to 1.00 wherein X and Y are as described above and Y′ represents the number of bases of the intervening sequence.

5. The testing chip according to claim 3, wherein a second primer contained in the amplification reagent comprises Sequence (Cc′ ) at the 3′ end portion which hybridizes to Sequence (C) at the 3′ end portion of a complementary sequence of the target nucleic acid sequence, and Sequence (D′ ) at 5′ side of the Sequence (Cc′ ) which hybridizes to Sequence (Dc) complementary to Sequence (D) present closer to 5′ side than the Sequence (C) in the complementary sequence of the target nucleic acid sequence.

6. The testing chip according to claim 5, wherein in the second primer, in the case where an intervening sequence is not present between the Sequence (Cc′ ) and the Sequence (D′ ), (X−Y)/X is in a range of −1.00 to 1.00 wherein X represents the number of bases of the Sequence (Cc′ ) and Y represents the number of bases of a region sandwiched between the Sequence (C) and the Sequence (D), and in the case where an intervening sequence is present between the Sequence (Cc′ ) and the Sequence (D′ ) in the primer, {X−(Y−Y′ )}/X is in a range of −1.00 to 1.00 wherein X and Y are as described above and Y′ represents the number of bases of the intervening sequence.

7. The testing chip according to claim 3, wherein a second primer contained in the amplification reagent comprises Sequence (Cc′ ) at the 3′ end portion which hybridizes to Sequence (C) at the 3′ end portion of a complementary sequence of the target nucleic acid sequence, and a folded sequence (D-Dc′ ) having two nucleic acid sequences which hybridize to each other on the same strand at 5′ side of the Sequence (Cc′ ).

8. The testing chip according to claim 7, wherein the folded sequence (D-Dc′ ) in the second primer is 2 to 1000 nucleotides in length.

9. The testing chip according to claim 1, wherein the amplification reagent is immobilized by a water-absorbing material.

10. The testing chip according to claim 1, wherein the amplification reagent is immobilized by a covalent bond.

11. The testing chip according to claim 1, wherein the test region further comprises a signal generation means which generates a signal derived from an amplification product.

12. The testing chip according to claim 11, wherein the signal is detectable by visual inspection.

13. The testing chip according to claim 1, which further comprises, between the sample introduction part and the test region in the inside or on the surface of the support, a part for immobilizing a nucleic acid extraction reagent wherein a nucleic acid extraction reagent is immobilized for extracting a nucleic acid from a liquid sample.

14. The testing chip according to claim 13, wherein the nucleic acid extraction reagent is a reagent for alkali extraction, a reagent for protease reaction, a surfactant or a combination thereof.

15. The testing chip according to claim 13, which further comprises, between the part for immobilizing the nucleic acid extraction reagent and the test region, a part for immobilizing a pH regulating reagent in the inside or on the surface of the support, wherein the pH regulating reagent is immobilized in order to make pH of a reaction solution for nucleic acid amplification formed in the test region suitable for an amplification reaction by the amplification reagent.

16. The testing chip according to claim 1, which is in a form of a test specimen, a sheet or a tape.

17. The testing chip according to claim 1, which comprises a plurality of test regions for amplifying a plurality of kinds of target nucleic acids individually.

18. The testing chip according to claim 1, wherein the target nucleic acid has a nucleic acid sequence specific to a wild-type gene or a variant-type gene or a pathogen.

19. The testing chip according to claim 16, wherein the pathogen is a virus, a bacterium or a fungus.

20. A method of detecting a target nucleic acid from a liquid sample using the testing chip according to claim 1, comprising steps of:
(a) bringing a liquid sample into contact with the sample introduction part on the testing chip;
(b) incubating the testing chip under conditions suitable for an amplification reaction of a nucleic acid by an amplification reagent contained in the test region; and
(c) detecting a signal from a nucleic acid amplification product of the amplification reaction of the nucleic acid.

21. The method according to claim 20, wherein the target nucleic acid has a nucleic acid sequence specific to a wild-type gene or a variant-type gene or a pathogen.

22. The method according to claim 21, wherein the pathogen is a virus, a bacterium or a fungus.

23. A method of analyzing a gene using the testing chip according to claim 1, comprising steps of:
(a) bringing a liquid sample into contact with the sample introduction part on the testing chip,
(b) incubating the testing chip under conditions suitable for an amplification reaction of a nucleic acid by an amplification reagent contained in the test region;
(c) detecting a signal from a nucleic acid amplification product by the amplification reaction of a nucleic acid;
(d) inputting the detected signal into a computer for gene analysis;
(e) comparing the signal with information available by the computer in the computer to characterize the signal and/or to search the information related to the signal; and
(f) outputting the characteristics of the signal and/or the information related to the signal from the computer.

24. The method according to claim 23, wherein the input into the computer in step (d) and the output from the computer in step (f) are carried out via a communication network.

25. The method according to claim 23, wherein the input into the computer in step (d) and the output from the computer in step (f) are carried out via a portable telephone connected to the communication network.


SEQUENCE LISTING (11 sequences)

SEQ ID NO: 1 - DNA, 38 bp, Artificial Sequence
  Description of Artificial SequencePrimer
ggatatatatatatccactgaacaaatgccacataaag

SEQ ID NO: 2 - DNA, 30 bp, Artificial Sequence
  Description of Artificial SequencePrimer
gcagcatcaccaacccaaaagcactgagta

SEQ ID NO: 3 - DNA, 28 bp, Artificial Sequence
  Description of Artificial SequencePrimer
cagtgtatgcgggagtggccgggagttg

SEQ ID NO: 4 - DNA, 29 bp, Artificial Sequence
  Description of Artificial SequencePrimer
gaagtgaaaacctgagcccccagcaggtc

SEQ ID NO: 5 - DNA, 28 bp, Artificial Sequence
  Description of Artificial SequencePrimer
tagtgtatgcgggagtggccgggagttg

SEQ ID NO: 6 - DNA, 29 bp, Artificial Sequence
  Description of Artificial SequencePrimer
aaagtgaaaacctgagcccccagcaggtc

SEQ ID NO: 7 - DNA, 44 bp, Artificial Sequence
  Description of Artificial SequencePrimer
gataaaacgccgcagacacatccttccaacctcttgtcctccaa

SEQ ID NO: 8 - DNA, 46 bp, Artificial Sequence
  Description of Artificial SequencePrimer
cctgctgctatgcctcatcttctttgacaaacgggcaacatacctt

SEQ ID NO: 9 - DNA, 156 bp, Homo sapiens
aagcttttaaagcatcctcattttatgtccaacatcagagacttaatactgaacaaatgccacataaaggtaatgactgttgaagaagatttaacttaacatcttgcagcatcactaagaactcgctttatactcagtgcttttgggttgggtttg

SEQ ID NO: 10 - DNA, 100 bp, Homo sapiens
gatgtcggggagtggccgggagttgggcgagtacgggctgcaggcatacactgaagtgaaaactgtgagtgtgggacctgctgggggctcagggcctgtt

SEQ ID NO: 11 - DNA, 149 bp, Hepatitis B virus
actcaccaacctcttgtcctccaacttgtcctggctatcgctggatgtgtctgcggcgttttatcatattcctcttcatcctgctgctatgcctcatcttcttgttggttcttctggactaccaaggtatgttgcccgtttgtcctcta